(19) United States
(12) Patent Application Publication
(10) Pub. No.: US 2024/0293033 A1
(43) Pub. Date: Sep. 5, 2024

(54) SENSORS CATHETER AND SIGNAL PROCESSING FOR BLOOD FLOW VELOCITY ASSESSMENT

(71) Applicant: MEDYRIA AG, Winterthur (CH)

(72) Inventor: Mauro Massimo SETTE, Winterthur (CH)

(21) Appl. No.: 18/576,505

(22) PCT Filed: Jul. 4, 2022
(86) PCT No.: PCT/EP2022/068365

§ 371 Date: Jan. 4, 2024

(30) Foreign Application Priority Data
Jul. 7, 2021 (DE) 10 2021 117 575.5

(51) Int. Cl. A61B 5/02; A61B 5/00; A61B 5/0215

(52) U.S. Cl. CPC A61B 5/02028; A61B 5/02158; A61B 5/7264; A61B 5/746

(57) ABSTRACT

An arrangement for measuring a flow velocity in a blood vessel comprising; a catheter configured to be inserted into a blood vessel; a plurality of flow velocity sensors coupled to the catheter; a sensor network coupled to the plurality of flow velocity sensors; and a processor coupled to the sensor network; wherein each of the plurality of flow velocity sensors is configured to sense a velocity of a blood flow, wherein an output of the sensor network is configured to be input into a mathematical model stored in the processor, and wherein the mathematical model is configured to calculate the flow velocity in the blood vessel where the catheter is located.

The present invention relates to an arrangement for measuring a flow velocity in a blood vessel and an associated method. The arrangement comprises a catheter, a plurality of flow velocity sensors coupled to the catheter, a sensor network coupled to the plurality of flow velocity sensors and a processor coupled to the system network.

### BACKGROUND TO THE INVENTION

There is a class of surgical procedures called interventional procedures or minimal invasive procedures, which comprise the introduction of a catheter within the human vasculature to measure quantities such as pressure and blood flow velocity. In a common procedure, the catheter is introduced from an opening in a blood vessel, e.g. in the groin or in the brachial artery. Through this opening the catheter is then advanced to the region of interest through the blood vessel, often to coronary branch. The interventional procedure is then performed at the region of interest.

Two parameters commonly measured by the catheter are pressure and blood flow velocity, which are then processed to calculate the two indexes called fractional flow reserve (FFR), and coronary flow velocity reserve (CFVR).

Both pressure-derived myocardial fractional flow reserve (FFR) and coronary flow velocity reserve (CFVR) have been evaluated as predictors of inducible ischemia, as measured by non-invasive stress tests, and indicate adverse events after stent placement.

The combination of pressure and flow velocity into an index of hyperemic stenosis resistance significantly improves diagnostic accuracy as assessed by noninvasive ischemic testing, especially in cases with discordant outcomes between traditional parameters.

The relationship between distal coronary velocity and trans-stenotic pressure gradient is almost entirely determined by the coronary stenosis and is thus by definition well suited to evaluate its hemodynamic severity.

In all the vascular procedures, such as stenting and ballooning of stenosis e.g. coronary or peripheral procedures, the flow velocity measurement and pressure measurements can always be used as diagnostic tool or for monitoring success of procedures.

A commonly used technology for measuring the blood flow velocity and its derived indexes is ultrasound. The catheters are equipped with a piezoelectric crystal that, when excited, can emit ultrasound. The ultrasound is then reflected by the natural blood scatters and the measurements of the Doppler frequency shift or the time of flight are used to derive the fluid's velocity.

For example, in order to describe the fluid flow in a blood vessel, both pressure and flow velocity may be necessary. An accurate knowledge of both pressure and flow velocity leads to a complete characterization of the fluid flow and to a definition of important diagnostic quantities such as the peripheral vessel's impedance.

For example, combining blood flow velocity and pressure measurement may be used as part of a guidewire for assessing a level of a stenosis.

The measurement made with an ultrasound/doppler sensor may be subject to the angle of incidence of the ultrasonic wave with the direction of the blood flow velocity according to the formula:

$${f}_{d} = - \frac{2 {f}_{s} v}{c} cos θ$$

where:
f_{d}=frequency shift
fₛ=frequency of the source
v—fluid's velocity
c—velocity of sound
cos θ—angle between direction of the velocity and direction of the emitted sound

Consequently, the measurement is strongly dependent on the angle θ which cannot be controlled in an endovascular procedure, i.e. the angle θ depending on the catheter position can range from 0° to 90° giving completely different measurements.

A common problem is a repeated injection of intracoronary adenosine as a signal of an instantaneous blood flow velocity cannot be measured with sufficient accuracy to rely on a mean blood flow velocity.

In WO 2019/149954 A1, an arrangement of sensors for providing information of an alignment of a catheter (in a blood vessel) is described. The content of WO 2019/149954 A1 is incorporated by reference herewith.

In this application a method for providing the sensor alignment within a vessel with respect to the yaw orientation is described (see z-axis of FIG. 1).

In the application, it was assumed that the pitch orientation can be neglected due to the radial-symmetry of a blood vessel. The identification of only the yaw orientation may not be sufficient to characterize the flow in the space because the information provided is only about the plane identified by the sensor and the information about the third dimension is missing. Thus, the roll information represents an important degree of freedom that must be taken into account.

E.g. the catheter can be in an optimal position (FIG. 2, Roll=0°) where the sensor's plane is aligned with the flow, or in a tilted position (FIG. 3, Roll=90°) where the sensor is placed in a region of minimum velocity (cf. FIG. 4 distribution of velocity around a cylinder). These positions may give very different flow velocity readings.

A possible solution to this problem could be a manual correction by the operator, where the operator manually rotates the catheter and therefore will be able to orientate it with the flow and obtain the measurement that will allow an optimal estimation of the flow.

This manipulation is not always doable since the catheter stiffness does not allow for 1:1 control of the distal tip when the catheter is manipulated proximally. The result will be a stick-slip type of rotation that may not allow for the optimal placement of the catheter. Moreover, the number of manipulations in the vessel may preferably be minimized in order to reduce the risk of plaque dislodgment within the vessel.

Moreover, the flow velocity distributions around a cylindrical shape is observed in FIG. 5, one can observe that in the boundary layers:
of the surface facing the flow the velocity is very low, because in this region the flow is stopped by the cylinder.
of the surfaces above and below, the velocity is higher, since that the flow gets accelerated by the cylinder.
of the surface after the cylinder the flow is very low because it is shadowed by the cylinder itself.

This means that for a single sensor placed on the surface of a catheter that is not perfectly aligned with respect to the flow, it is difficult to measure the real vessel flow velocity, due to the fact of having a non-negligible geometry in the flow will in any case either accelerate or decelerate the flow velocity which may produce an altered measurement.

Arrangements may have to be optimized with respect to positioning of sensors on catheters. Nevertheless, it is desired to form an arrangement enabling better measuring accuracy.

There is therefore a demand for providing concepts for equipping catheters with sensor arrangements that allow for an improved measurement of blood flow velocity, thereby giving accurate information on a correct positioning of a catheter.

Such a demand may be satisfied by the subject-matter of the claims.

### SUMMARY OF THE INVENTION

The invention is set out in the independent claims. Preferred embodiments of the invention are outlined in the dependent claims.

According to a first aspect, an arrangement for measuring a flow velocity in a blood vessel is described. The arrangement comprises a catheter configured to be inserted into a blood vessel, a plurality of flow velocity sensors coupled to the catheter, a sensor network coupled to the plurality of flow velocity sensors and a processor coupled to the sensor network. Each of the plurality of flow velocity sensors is configured to sense a velocity of a blood flow. An output of the sensor network is configured to be input into a mathematical model stored in the processor. The mathematical model is configured to calculate the flow velocity in the blood vessel where the catheter is located.

The catheter may be any commercially available catheter which is configured to be inserted into a blood vessel. The catheter may comprise any suitable material such as, for example, PVC and/or rubber and/or silicon. The catheter may preferable be of such a design that allows for a plurality of flow velocity sensors to be coupled to or arranged on said catheter. The catheter is preferably cylindrical in its cross-section but may alternatively be cuboidal, triangular or a bespoke shape. In some examples, the catheter is instead an elongated body or element which may not be suitable to be used as a catheter. A blood vessel is described here and within this description. However, the catheter/elongated body or element may be inserted into any channel through which a fluid is flowing such as, for example, air and/or water and/or oil.

The flow velocity sensors may be sensors which are configured to measure the velocity of the blood flow in a vector format. The vector format preferably comprises three components which correspond to an x-, a y- and a z-axis of the sensor. The plurality of flow velocity sensors may be coupled to the sensor network so as to form a part of the sensor network. In other words, the plurality of flow velocity sensors may be configured to be a part of the sensor network. The flow velocity sensors may be tilted together with the catheter (θ>0°) with respect to the blood flow velocity. For example, when the catheter is tilted with an angle > or < than 0°, the two measurements may influence each other, i.e. one sensor will exchange power with a slightly warmed up fluid and then the two measurements will be different. These measurements may then be individually transmitted to the sensor network or may be combined before transmission to the sensor network.

The sensor network is coupled to the plurality of flow velocity sensor and comprises components which allow for data relating to the flow velocity to be transmitted. The plurality of flow velocity sensors may be coupled to the sensor network via a wired coupling or a wireless coupling. If the coupling is a wired coupling, the wire coupling the components preferably has a small diameter so as to not disrupt the reading from the flow velocity sensors. If the data from the flow velocity sensors are transmitted to the sensor network individually, the sensor network may combine the received data measurements.

The processor is coupled to the sensor network and is configured to receive the data transmitted by the sensor network. The processor may be able to calculate the flow velocity via the mathematical model. The processor may be coupled to a memory which may be configured to store results of the processor and/or store the mathematical model which the processor uses. If the sensor network does not combine the received data measurements from the plurality of flow velocity sensors, the processor may combine these measurements before using said measurements in the model. In some examples, the received measurements are not used in a model.

In some examples, the sensor network further comprises a pressure sensor, wherein the pressure sensor is configured to sense a pressure within the blood vessel. The combination of flow velocity sensors and a pressure sensor may allow for an index of hyperemic stenosis resistance. The pressure sensor may be a piezoelectric pressure sensor and/or an optical pressure sensor based on the Fabry-Perot interferometer principle. The signal from the pressure sensor may be used as an input for the mathematical model described in the present description. If the signal of the pressure sensor is used in combination with the flow velocity sensor, this may result in a measurement of the peripheral/vascular resistance which may be particularly advantageous in clinical uses. This may in turn may significantly improve diagnostic accuracy as assessed by noninvasive ischemic testing, especially in cases with discordant outcomes between traditional parameters

In some examples, the mathematical model comprises or is a function, a polynomial function, a regression model, a lumped parameter model, a decision tree, a random forest, a neural network or a numerical model, wherein the mathematical model is configured to output a velocity vector of the flow velocity. The mathematical function may be chosen based on the parameters to be measured by the flow velocity sensors and/or the environment the catheter is in.

In some examples, the velocity vector is independent of the catheter orientation. This may allow for the measurement of the blood flow velocity regardless of the orientation of the flow velocity sensors. This may allow for a more accurate measurement of the blood flow velocity.

In some examples, a quality of the sensed parameters is configured to be evaluated by means of at least one of a regression coefficient, a correlation coefficient, or a fitting coefficient. This may allow for the processor to compare the result from the flow velocity sensors with an expected result from the mathematical model. The quality of the result may then be transmitted to a display screen which may be seen by a user which may then allow them to alter the positioning of the catheter based on the quality indicated.

In some examples, the mathematical model comprises information about a geometry of the catheter and/or an impact of the catheter on the flow, wherein the information is configured to allow for the mathematical model to compensate for the geometry of the catheter and/or the impact of the catheter on the flow. This may allow for a more accurate measurement of the blood flow as the disruption to the blood flow caused by the catheter being in the blood vessel can be reduced and can be accounted for within the mathematical model.

In some examples, an output of the mathematical model is signalled to a user. This signalling may be via a system of LEDs and/or via a display screen. The output displayed may be the blood flow velocity and/or pressure within the blood vessel. This may allow for a safer operation as the user can abort the procedure if the blood flow velocity and/or pressure falls outside of a predetermined parameter.

In some examples, the mathematical model is configured to be tailored to be specific for different blood vessel geometries and flow conditions. This may allow for a more accurate measurement of the blood flow. In particular, the mathematical model may be altered based on the diameter of the blood vessels (e.g. aortas or coronaries) and/or based on the situation of the retrograde flow (e.g. arterial vs venous vessels).

In some examples, the mathematical model is tuned to identify laminar and/or transition and/or and turbulent flow regimes. The mathematical model may be tuned by altering the parameters and/or hyperparameters of the model being used. The mathematical model may be tuned by an experimental method where a series of benchmark tests are undertaken followed by the results of these tests being used to tune the mathematical model. In some examples, the results are input into a regression algorithm and the results of these algorithms are used to tune the model. Additionally or alternatively, the mathematical model may be tuned by an experimental method were different simulations may be run and then the parameters are extracted from these simulations. These parameters may then be used to tune the model. Additionally or alternatively, the mathematical model may be tuned by a machine learning method. The use of machine learning is known to the skilled person. This may allow for a more accurate blood flow velocity measurement and/or if there is a problem during the intervention. This may then allow the user to abort the intervention thereby leading to a safer intervention process.

In some examples, the plurality of flow velocity sensors are hot-wire anemometer sensors. A hot-wire anemometer sensor may be particularly useful in situations where there are turbulent flows, they may also allow for an analogue output which may provide an opportunity for conditionally-sampled time-domain and frequency-domain analysis and/or for the measurement of multi-component flows.

In some examples, each of the plurality of flow velocity sensors are configured to thermally influence at least one other flow velocity sensor in the plurality of flow velocity sensors. The alignment of the catheter with respect to the blood flow may be determined by using thermal cross talk between the plurality of sensors. For example, when the catheter is correctly aligned with the flow velocity stream i.e. the angle with respect to the blood flow is 0°, the plurality of sensors may provide the same measurement. This may allow for a more accurate blood flow velocity measurement.

In some examples, the mathematical model is a numerical model, and wherein the mathematical model comprises a Navier-Stokes equation, wherein an output of the Navier-Stokes equation is compared with the sensed blood flow velocity, and wherein an index of merit is configured to be calculated based on the comparison. This may allow for the mathematical model to be modelled particularly for viscous fluids, thereby allowing for a more accurate measurement of the blood flow velocity. The index of merit may allow for the user to see whether the calculated blood flow velocity is reliable, thereby maintaining the safety of the intervention.

In some examples, the mathematical model is a lumped parameter model, wherein the lumped parameter model comprises or consists of discrete entities configured to approximate the behaviour of the output of the plurality of flow velocity sensors, and wherein the lumped parameter model is defined by

$$\frac{dQ}{dt} = - h \cdot A ( T ( τ ) - {T}_{env} ) = - h \cdot A Δ T ( t ) ,$$

wherein Q is thermal energy in Joules, h is a heat transfer coefficient between the catheter and the blood flow, A is surface area of the heat transfer, T is a temperature of a surface of the catheter, Tₑₙᵥ is a temperature of the environment and ΔT(t) is a time-dependent thermal gradient between the environment and the catheter. This may allow for an improved blood flow velocity measurement and/or improved user safety as the results of the calculations can easily be shown to a user.

In some examples, the arrangement further comprises an alarm, wherein the alarm indicates to a user if the blood flow velocity falls outside of a predetermined range.

This may improve the safety of the intervention as the user can easily be informed if there is a problem with the intervention procedure.

According to a second aspect, a method for measuring a flow velocity in a blood vessel by an arrangement is described. The arrangement comprises a catheter configured to be inserted into a blood vessel, a plurality of flow velocity sensors coupled to the catheter, a sensor network coupled to the plurality of flow velocity sensors and a processor coupled to the sensor network. The method comprises sensing a velocity of a blood flow of the blood vessel by each of the plurality of flow velocity sensors, inputting an output of the sensor network into a mathematical model stored in the processor and calculating, by the mathematical model, the flow velocity in a blood vessel where the catheter is located.

The catheter may be any commercially available catheter which is configured to be inserted into a blood vessel. The catheter may comprise any suitable material such as, for example, PVC and/or rubber and/or silicon. The catheter may preferable be of such a design that allows for a plurality of flow velocity sensors to be coupled to said catheter. The catheter is preferably cylindrical in its cross-section but may alternatively be cuboidal, triangular or a bespoke shape.

The flow velocity sensors may be sensors which are configured to measure the velocity of the blood flow in a vector format. The vector format preferably comprises three components which correspond to an x-, a y- and a z-axis of the sensor. The plurality of flow velocity sensors may be coupled to the sensor network so as to form a part of the sensor network. In other words, the plurality of flow velocity sensors may be configured to be a part of the sensor network.

The sensor network is coupled to the plurality of flow velocity sensor and comprises components which allow for data relating to the flow velocity to be transmitted. The plurality of flow velocity sensors may be coupled to the sensor network via a wired coupling or a wireless coupling. If the coupling is a wired coupling, the wire coupling the components preferably has a small diameter so as to not disrupt the reading from the flow velocity sensors.

The processor is coupled to the sensor network and is configured to receive the data transmitted by the sensor network. The processor may be able to calculate the flow velocity via the mathematical model. The processor may be coupled to a memory which may be configured to store results of the processor and/or store the mathematical model which the processor uses.

In some examples, not all of these steps are required. In some examples, the steps may be in a different order. In some examples, some of the steps happen simultaneously.

It is clear to a person skilled in the art that the statements set forth herein may be implemented under use of hardware circuits, software means, or a combination thereof. The software means can be related to programmed microprocessors or a general computer, an ASIC (Application Specific Integrated Circuit) and/or DSPs (Digital Signal Processors). For example, the processing unit may be implemented at least partially as a computer, a logical circuit, an FPGA (Field Programmable Gate Array), a processor (for example, a microprocessor, microcontroller (μC) or an array processor)/a core/a CPU (Central Processing Unit), an FPU (Floating Point Unit), NPU (Numeric Processing Unit), an ALU (Arithmetic Logical Unit), a Coprocessor (further microprocessor for supporting a main processor (CPU)), a GPGPU (General Purpose Computation on Graphics Processing Unit), a multi-core processor (for parallel computing, such as simultaneously performing arithmetic operations on multiple main processor(s) and/or graphical processor(s)) or a DSP.

It is further clear to the person skilled in the art that even if the herein-described details will be described in terms of a method, these details may also be implemented or realized in a suitable device, a computer processor or a memory connected to a processor, wherein the memory can be provided with one or more programs that perform the method, when executed by the processor. Therefore, methods like swapping and paging can be deployed.

### BRIEF DESCRIPTION OF THE DRAWINGS

Even if some of the aspects described above have been described in reference to the arrangement, these aspects may also apply to the method and vice versa.

These and other aspects of the invention will now be further described, by way of example only, with reference to the accompanying figures, wherein like reference numerals refer to like parts, and in which:

FIG. 1 shows a three-axis geometry according to the prior art;

FIGS. 2 and 3 show views of a catheter and a flow velocity sensor according to the prior art;

FIGS. 4 and 5 show velocity distributions around a cylinder according to the prior art;

FIG. 6 shows a view of a catheter and flow velocity sensors according to an embodiment described herein;

FIG. 7 shows a view of a catheter, flow velocity sensors and a sensor network according to an embodiment described herein;

FIG. 8 shows a flow diagram of a data flow according to an embodiment described herein;

FIG. 9 shows a perspective view of a catheter according to an embodiment described herein;

FIG. 10 shows cutaway views of catheters according to an embodiment described herein;

FIG. 11 shows a flow diagram of a data flow according to an embodiment described herein; and

FIG. 12 shows a block diagram of a method for measuring a flow velocity in a blood vessel according to an embodiment as described herein.

### DETAILED DESCRIPTION OF THE PREFERRED EMBODIMENTS

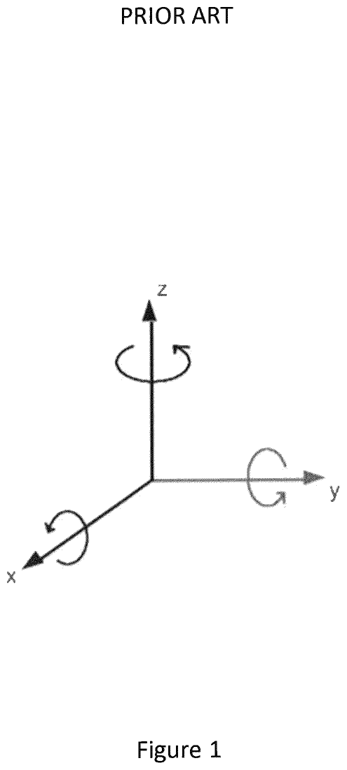

FIG. 1 shows a three-axis geometry according to the prior art.

In the prior art, the flow of blood within a vessel is primarily along the x-axis. The blood flow velocity is measured by a flow velocity sensor with respect to the yaw orientation of the blood vessel i.e. about the z-axis. The prior art does not consider the pitch orientation i.e. about the y axis, due to the radial-symmetry of a blood vessel. Therefore, the roll information represents an important degree of freedom that must be taken into account.

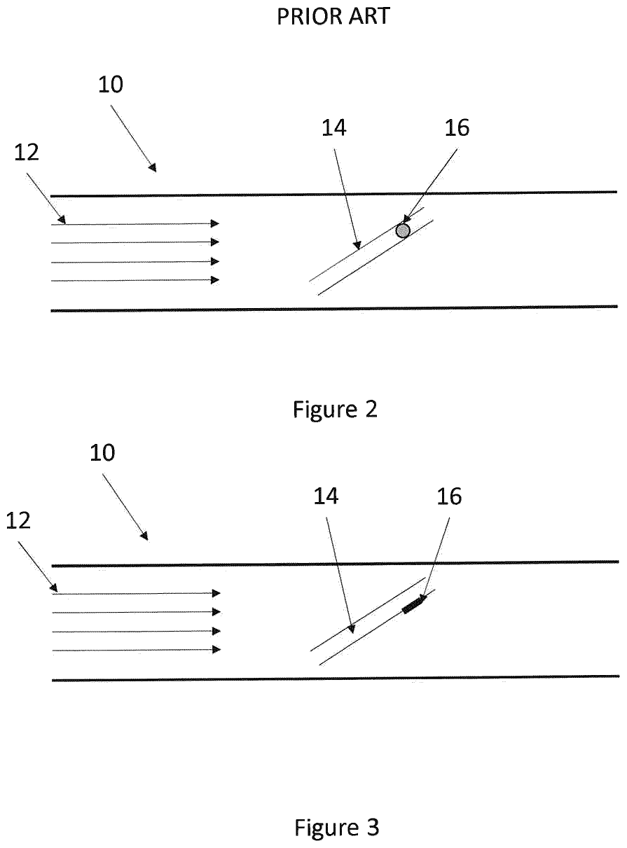

FIGS. 2 and 3 show views of a catheter and a flow velocity sensor according to the prior art.

The blood vessel 10 comprises a blood flow in a direction substantially the same as the direction of the arrows 12. Situated within the blood vessel 10 is a catheter 14 with a single flow velocity sensor 16 coupled to the catheter 14. The flow velocity sensor 16 is configured to sense the velocity of the blood flow within the blood vessel 10.

The catheter 14 can be in an optimal orientation, as shown in FIG. 2, when the roll angle is 0°. In this optimal orientation, the main plane of the flow velocity sensor 16 is aligned with the blood flow. Alternatively, the catheter 14 may be in a suboptimal orientation, as shown in FIG. 3, when the roll angle is 90°. In this suboptimal orientation, the main plane of the flow velocity sensor 16 is placed in a region of minimum velocity i.e. hidden behind the catheter 14. Furthermore, as the flow velocity sensor 16 is behind the catheter 14, the flow velocity sensor may not accurately measure the velocity of the blood flow within the blood vessel 10.

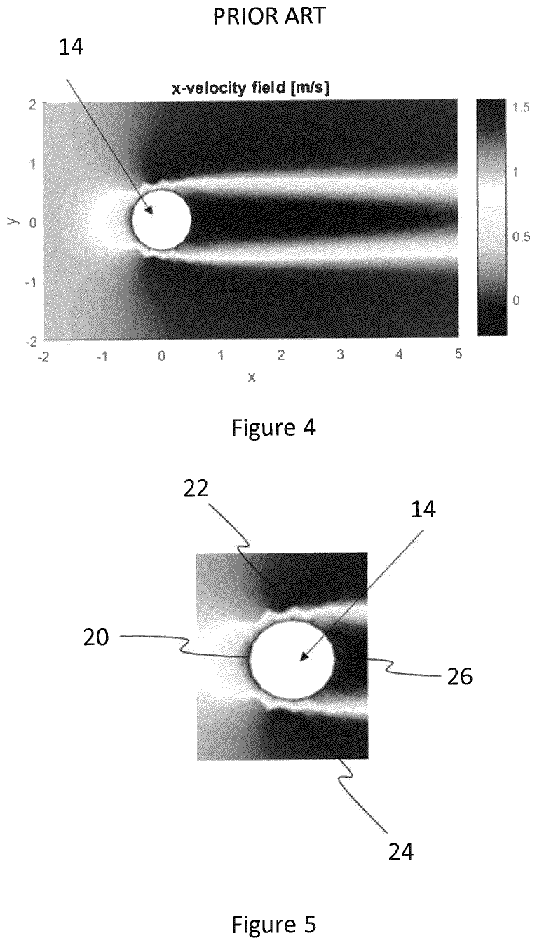

FIGS. 4 and 5 show velocity distributions around a cylinder according to the prior art.

FIG. 4 shows a velocity field when the catheter 14 is inside a blood vessel 10.

Moreover, the flow velocity distributions around a cylindrical shape can be observed in FIG. 5, one can observe that in the boundary layers:
of the surface 20 facing the flow, the velocity is very low, because in this region the flow is stopped by the cylinder.
of the surfaces 22, 24 above and below the catheter 14, the velocity is higher, since the flow gets accelerated by the catheter 14.
of the surface 26 after the catheter 14, the flow velocity is very low because it is shadowed by the catheter 14 itself.

This type of fluid dynamics is known to the skilled person. In the above, this means that for a single flow velocity sensor 16 placed on a surface of the catheter 14 that is not perfectly aligned with respect to the flow, it is difficult to measure the real flow velocity. This is due to the fact that the catheter 14 has a non-negligible geometry in the flow and the catheter 14 will in any case either accelerate or decelerate the flow velocity due to fluid dynamics.

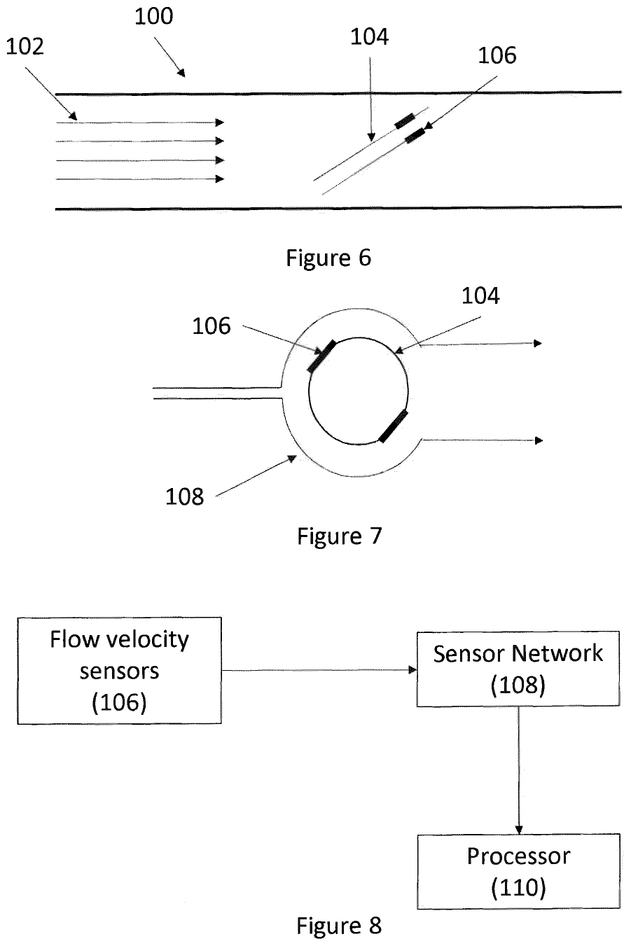

FIG. 6 shows a view of a catheter and flow velocity sensors according to an embodiment described herein.

FIG. 6 shows a blood vessel 100 with the blood flow within the blood vessel 100 moving in a direction substantially parallel to the arrows 102. A catheter 104 is situated within the blood vessel 100. Two flow velocity sensors 106 are directly coupled to the catheter 104. The flow velocity sensors 106 may be able to sense the velocity of the blood flow by pressure, wherein the pressure exerted on the flow velocity sensors 106 by the blood flow may allow for the flow velocity to be calculated. The flow velocity sensors 106 may comprise an electronic circuit which allows for the flow velocity sensors 106 to calculate the flow velocity sensed by that sensor 106. In this embodiment, the two flow velocity sensors 106 are coupled to the catheter 104 on opposite sides of the catheter 104 i.e. the sensors 106 are situated 180° from each other. In some embodiments, the flow velocity sensors 106 are not situated 180° to each other and may be any suitable angle away from each other such as, for example, 45°, 90° or 120°. In some embodiments, there are more than two flow velocity sensors 106. In embodiments with more than two flow velocity sensors 106, the flow velocity sensors 106 may be equally spaced from each other. For example, if there are three flow velocity sensors 106, the sensors 186 may be spaced 120° apart from each other. Alternatively, the sensors 106 may be placed at any suitable distance from each other.

FIG. 7 shows a view of a catheter, flow velocity sensors and a sensor network according to an embodiment described herein.

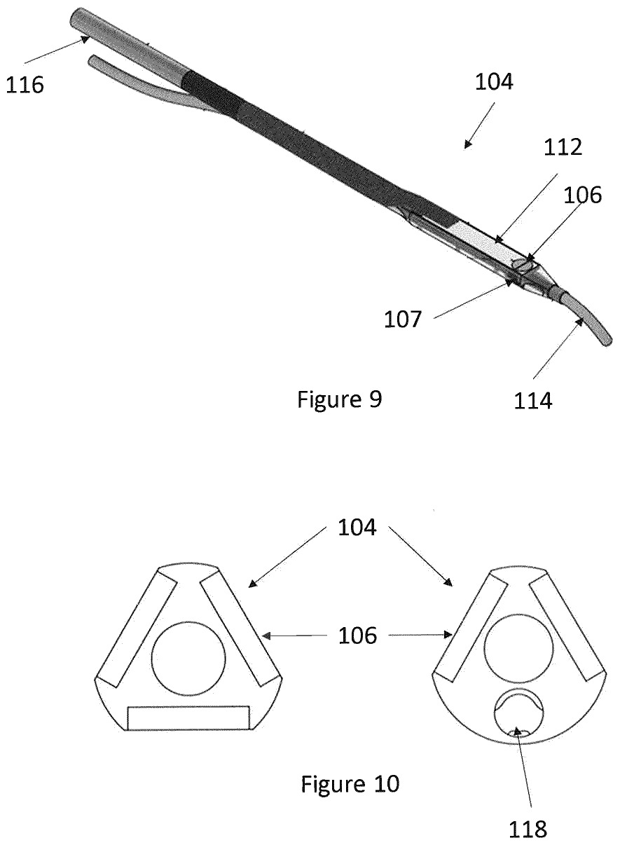

FIG. 7 shows a sensor network 108 coupled to the flow velocity sensors 106 on the catheter. The coupling between the flow velocity sensors 106 and the sensor network 108 may be a wired coupling or a wireless coupling. In a wireless coupling, each flow velocity sensor 106 may comprise a transmitter which is configured to transmit its sensed reading to the sensor network 108, and the sensor network 108 is configured to receive the transmitted readings. In this embodiment, the sensor network 108 may be located in, or coupled to, a computer and/or a processor (see FIG. 8) and/or located in cloud storage. The sensor network 108 comprises two or more sensors 106 integrated within a sensor housing (see FIG. 9). It is particularly preferable that the sensors 106 are located around the catheter 104 in a symmetric manner. For example, if the catheter 104 has three sensors 106, the sensors 106 are preferably placed 120° apart. This is particularly advantageous for the measurement of flow velocity.

In a wired coupling, the sensor network 108 may comprise electrical wires coupled to each flow velocity sensor 106, wherein the electrical wires are configured to transmit the sensed readings of the flow velocity sensors 106 to, for example, a computer and/or a processor. The electrical wires may also be coupled to the catheter 104 so that they do not come loose within the blood vessel 100 and/or to allow for a minimal disruption in the fluid dynamics around the catheter. Additionally or alternatively, the electrical wires may be located within the catheter 104.

The sensor network 108 may also allow for the sensed readings from the plurality of flow velocity sensors 106 to be collated into a single three-dimensional velocity vector. In some embodiments, the sensor network does not collate the sensed readings and keeps them in separate data flows.

For ease of illustration, FIG. 7 shows the catheter in a cut-away view. The skilled person understands that the sensor network 108 may be in any suitable orientation with respect to the flow velocity sensors 106 and the catheter 104.

In some examples, there may be additional sensors. For example, there may be a pressure sensor and/or a sensor which senses collisions with a wall of the blood vessel 100 and/or any other suitable sensor configured to aid intervention.

FIG. 8 shows a flow diagram of a data flow according to an embodiment described herein.

FIG. 8 shows that the sensed readings from the flow velocity sensors 106 are transmitted to a processor 110 via the sensor network 108 which the flow velocity sensors 106 are part of. The processor 110 may comprise a processing unit. The processor may be coupled to a memory unit and/or a display unit as will be described in further detail below. The coupling between the flow velocity sensors 106 and the sensor network 108 may be wired or wireless, as described above. The coupling between the sensor network 108 and the processor 110 may also be wired or wireless. In a wired coupling, the electrical cables of the sensor network 108, as described above, may transmit the sensed readings directly to the processor 110. In a wireless coupling, the sensor network 108 may comprise a transmitter configured to transmit the sensed readings of the flow velocity sensors 106 and the processor 110 may comprise a receiver configured to receive the sensed readings from the sensor network 108.

FIG. 9 shows a perspective view of a catheter according to an embodiment described herein.

FIG. 9 shows a catheter 104 configured to be inserted into a blood vessel 104. On the catheter is a plurality of flow velocity sensors 106 and pressure sensors 107. The pressure sensors may be similar to the pressure sensors 107 described above. The sensors 106, 107 are located within a housing 112 located at the distal end of the catheter 104. The sensor housing 112 may comprise any material suitable for use in a catheter such as, for example, plastics. The housing 112 is preferably flexible to allow for greater ease of movement of the catheter 104. The sensor network 108 is preferably located within the housing 112. Located at the distal end of the catheter 104 is a tip 114 suitable for interventions. The sensor network 108 transmits the measured data from the plurality of sensors 106, 107 via a hypotube 116 to the processor 110 which is located at an external location. The hypotube 116 may alternatively be any type of tube suitable for transmitting data and for being used as part of a catheter 104. The remainder of the parts of the catheter 104 not mentioned above are known to the skilled person.

FIG. 10 shows cutaway views of catheters according to an embodiment described herein.

The catheter 104 preferably has sensors 106, 107 located at equidistant angles around the catheter 104. For example, if the catheter 104 has three sensors 106, 107, the sensors 106, 107 are preferably placed 120° apart. This is particularly advantageous for the measurement of flow velocity. In some embodiments, the catheter 104 comprises a channel 118. This channel 118 may preferably used for transmission of data from the sensors 106, 107 to the sensor network 108 and then to the processor 110. This channel 118 may alternatively be used for extra instruments which may be required to be used during an intervention such as, for example, a guide wire.

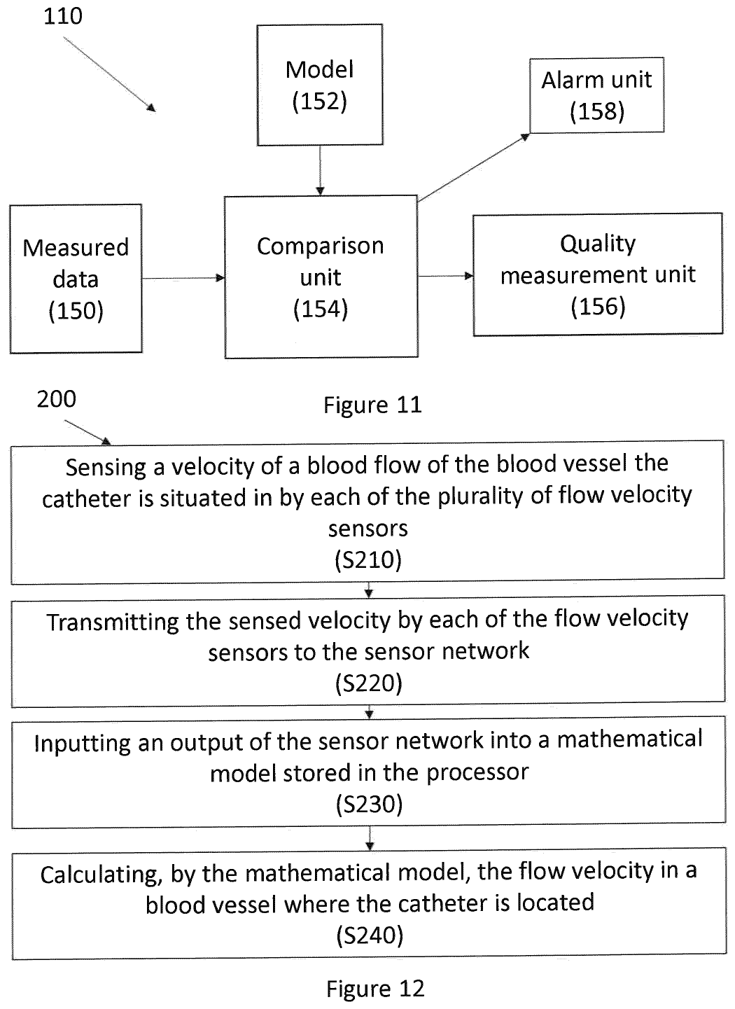

FIG. 11 shows a flow diagram of a data flow according to an embodiment described herein.

In the processor 110, the processor 110 receives the measures sensed data 150 from the flow velocity sensors 106 via the sensor network 108 as described above. The processor 110, either within the processor 110 itself or via a memory unit (not shown), receives a mathematical model 152. The mathematical model 152 may be able to estimate the expected flow velocity sensed by the flow velocity sensors 106 and/or may input the flow velocity sensed by the flow velocity sensors 106 into the model 152 itself.

The mathematical model 152 can be of different natures. For example, the model 152 may be a polynomial function where the coefficients are tuned to provide accurate flow measurements:

$$\vec{v} = f ( {x}_{1} , ... , {x}_{n} )$$

### Where:

f( ), is a polynomial function
x₁, . . . , xₙ: Are the different measurements inputs
\vec{v}: is the velocity vector

The velocity vector output by the polynomial function is a vector comprising 3 components, wherein each component relates to a different axis of the blood vessel i.e. the x-, y- and z-axes. In some embodiments, the velocity vector only has less than three components. The axes represented by this reduced vector may be altered based on the wish of a user.

The mathematical model 152 may be the polynomial function described above and/or may comprise at least one of the following mathematical models 152. In all of the following described mathematical models 152, the skilled person understands the limitations of each model 152 and the possible alterations that can be made to the said models 152:

### One Variable Regression

A feature is chosen that suitably describes the influence of the orientation of the catheter 104, wherein the feature is a feature where a correction is able to compensate for the fluid dynamics surrounding the catheter 104. The feature may be, for example, a quantity which represents the raw signals received from the sensors 106, 107. The feature may additionally or alternatively be a ration between the raw signals received from the sensors 106, 107. Equation 1 shows a general representation of a one variable regression equation, with the feature x, the target y and weights cᵢ to account for the fluid dynamics around the catheter 104, the equation may be used for the estimation of the flow velocity. The target may be, for example, the flow velocity within the blood vessel 100 and the weights may be the model parameters. The choice of the feature and the target (correction) as well as the order n of the model are possible hyperparameters. There are very few tuning possibilities for this model 152.

$$y - \text{?} \cdot \text{?} \qquad Equation 1$$

$$\text{?} \text{indicates text missing or illegible when filed}$$

### Multivariate Linear Regression

The multivariate linear regression is similar to the one variable regression. Equation 2 shows a general representation of a multivariate linear regression, with the i-th feature value xᵢ, target y and weights cᵢ. The target (correction), number m of features and the composition of these features are possible hyperparameters.

$$y = \text{?} \cdot \text{?} {c}_{intercept} \qquad Equation 2$$

$$\text{?} \text{indicates text missing or illegible when filed}$$

### Decision Tree

A decision tree algorithm comprises or consists of nodes, branches and leaves. During the fitting of the model 152, a comparison may established for each node. Depending on the values given, the decision follows one of the two branches to the next node. Finally, when reaching the leaf at the bottom of the decision tree, a decision is taken. Decision trees are very adjustable and may offer a variety of hyperparameters that can be tuned, as listed in Table 1 below.

TABLE 1
Overview of the available hyperparameters for decision trees.

| Hyperparameter | Description |
|---|---|
| max_depth | Maximum depth of the decision tree. |
| min_samples_split | Minimum number of samples needed to split a node. |
| min_samples_leaf | Minimum number of samples needed to create a leaf |
| | node. |
| max_features | Number of features considered when looking for the |
| | best split. |
| max_leaf_nodes | Maximum numbers of leaves created. |

Due to their adjustability and versatility, decision trees are able to predict a wide range of data. Furthermore, they can be used for classification as well as regression. However, this gives a risk of overfitting the model to the data. Additionally, the implementation and prediction time could become troublesome for big trees with a large depth and many nodes/leaves. The skilled person understands that the various parameters can be, for example, a blood flow velocity of a single component of the velocity vector and/or increasing or decreasing velocity and/or if a component of the velocity vector falls outside of a predetermined parameter.

### Random Forest

The random forest model is an ensemble method, which means that the estimation of multiple models is taken into account for the final estimation. In the case of the random forest the ensemble may comprise or consist of a defined amount of decision trees “n_estimators”. Each tree is defined individually on a sample of the full data “max_samples”. These two parameters are additional hyperparameters to the ones from the individual decision trees and can be used to adjust the model 152. Finally, each decision tree makes a prediction and the final result is the average of the individual estimations of the decision trees.

### Lumped Parameters

The lumped parameter physical model simplifies the complex physical phenomena into a topology comprising or consisting of discrete entities that approximate the behaviour of the distributed system. This model 152 may be defined by:

$$\frac{dQ}{dt} = - h \cdot A ( T ( τ ) - {T}_{env} ) = - h \cdot A Δ T ( t ) ,$$

wherein Q is thermal energy in Joules, h is a heat transfer coefficient between the catheter 104 and the blood flow, A is a surface area of the heat transfer, T is a temperature of a surface of the catheter 104, Tₑₙᵥ is a temperature of the environment and ΔT(t) is a time-dependent thermal gradient between the environment and the catheter 104.

### Deep Neural Network for Regression

A Neural Network is a collection of connected nodes or units, structured in layers. Each unit represents a non-linear function, which takes as the input, the outputs of the previous layer and provides as output, a weighted sum after applying a non-linear function i.e. an activation function. The final layer is the output layer and forgoes the non-linear functions as seen in the previous layers and outputs a weighted sum of its inputs.

For the calculation of the weights for each weighted sum, neural networks are trained by processing input-output examples in order to optimize a loss function. The neural networks use optimization algorithms based on gradient descent and back propagation to adjust the weights of all the nodes in every layer. This may allow for a particularly accurate deep-learning mathematical model.

### Recurrent Neural Network

A Recurrent Neural Network (RNNs) is a subclass of Neural Networks that takes into account previous outputs by having hidden states. Thus, it exhibits temporal dynamic behavior.

Gated Recurrent Units (GRUs) and Long Short-Term Memory units (LSTMs) are subtypes of RNNs that deal with the problem of the vanishing gradient, allowing them to capture long term dependencies.

For the training of such networks, back propagation is done at each point in time (Back propagation through time). Thus, all the points of the time sequence have an effect on the weights of each single unit.

This may allow for a particularly accurate model due to the improved deep-learning techniques.

### Numerical Model

The result of the numerical solution of the Navier-Stokes equation describing the flow velocity distribution around the catheter 104 is used to compare the measurements performed by the flow velocity sensors. An index of merit for assessing the fitting between the measurements and the model 152 defining the three dimensional flow velocity vector may be produced.

In addition to the above, the mathematical model 152 may also comprise a correction component configured to correct for the fluid dynamics around the catheter 104 and/or the influence of an unknown orientation of the flow velocity sensors 106 and/or catheter 104. The correction component may be at least one of the following:

### Power Correction

Looking at the influence of the yaw orientation on the power-velocity curve of the sensor samples may allow for a possible way for compensation of the yaw orientation. The yaw orientation may be compensated by adjusting the power values of the sensor 106 depending on the orientation of the flow velocity sensors 106 and/or catheter 104 so that the power is within a certain range independent of the orientation. There are 2 possible approaches to adjust the power. First, the power may be shifted up or down by adding/subtracting a power value depending on the orientation. Second, the power may be scaled by a factor that depends on the orientation.

In general, the required correction is defined by determining a representative power-velocity curve and calculating the needed shift/factor to move the measured value to the determined curve. As possible representative p-v curves, the minimum, mean and maximum values of the chosen dataset may be considered. However, due to the flattening behaviour of the p-v curve, the minimum is not considered to be a good choice because it brings a high risk that corrected values could be outside of the validity range.

Finally, the partition of the dataset, on which the representative curve is calculated, needs to be defined. Therefore, two main approaches are identified. Firstly, each sensor sample is considered individually. This means that the representative curve is calculated for each sensor sample. The advantage is that this neglects the difference between the sensor samples. However, it requires an individual calibration for each sensor 106 to determine the behavior of the sensor. Secondly, all sensors 106 are considered for the representative curve. The advantage is that this correction may generalize the behavious of the sensors 106 well. However, it may lead to a decrease in the accuracy if the differences between the samples are not taken into account.

### Evaluation of Measurements Accuracy

Regression coefficients, correlation indexes and indexes to evaluate the fitting of the data into the model can be used to evaluate the quality of the data. This may provide information about the quality of the measurements collected.

In this way, the system model-sensor network 108 will also be able to estimate non-optimal measurements, e.g. induced by a non-optimal exposure to the flow such as when the sensor 106 is touching a wall of the blood vessel 100. This non-optimal measurement may be deduced by comparing the different measurements in the network 108 and by defining indexes (e.g. correlation with models or regression indexes of the functions) that may show bad data quality.

The model 152 may be chosen by a user based on, for example, the diameter of the blood vessel 100, the location where the catheter 104 will be situated and/or the retrograde flow of the blood vessel 100. This alteration may be made by the user via a display coupled to the processor 110. Alternatively, the change in model 152 may be done automatically by the processor 110 in the form of a machine-learning algorithm within the processor 110.

After the model 152 has been chosen, the sensed readings 150 and then compared with the results of the model 152 in a comparison unit 154. The sensed readings 150 may also be put through the model 152 before the comparison is made.

The comparison unit 154 then sends data regarding this comparison to the quality measurement unit 156. The quality measurement unit 156 measures the quality of the sensed readings 150 in relation to the estimated velocity vector from the model 152. The quality measurement unit 156 then makes a decision on the quality of the sensed readings 150. This decision may then be shown in a display unit coupled to the processor 110 to indicate to the user the quality of the readings 150. The user may then make a decision regarding the intervention based on the quality of the readings 150.

In some embodiments, the processor further comprises an alarm unit 158. The alarm unit 158 may indicate to the user when a component of the velocity vector of the blood flow has fallen outside of a predetermined range and/or when the comparison unit 154 finds a large discrepancy between the model 152 and the sensed readings 150. The alarm may be an audio alarm and/or a haptic alarm and/or a visual alarm. If the alarm is a visual alarm, this may be shown on the display unit coupled to the processor 110.

FIG. 12 shows a block diagram of a method for measuring a flow velocity in a blood vessel according to an embodiment as described herein.

The method 200 for measuring a flow velocity in a blood vessel 100 comprises of four main steps.

First, a velocity of a blood flow of the blood vessel 100 is sensed S210 by each of the plurality of flow velocity sensors 106. This may allow for the flow velocity of the blood vessel 100 to be accurately determined.

Then, the sensed velocity by each of the flow velocity sensors 106 is transmitted S220 to the sensor network 108. This may allow for the sensed velocities to be collated into one reading.

An output of the sensor network 108 is then input S230 into a mathematical model 152 stored in the processor 100. This may allow for the sensed velocities 150 to be corrected for fluid dynamics irregularities and/or allow for the blood flow velocity within the blood vessel 100 to be more accurately defined.

The flow velocity in the blood vessel where the catheter 104 is located is then calculated S240 by the mathematical model 152. This may allow for an accurate reading of the blood flow velocity.

No doubt many other effective alternatives will occur to the skilled person. It will be understood that the invention is not limited to the described embodiments and en-compasses modifications apparent to those skilled in the art and lying within the scope of the claims appended hereto.

1. An arrangement for measuring a flow velocity in a blood vessel comprising:
a catheter configured to be inserted into a blood vessel;
a plurality of flow velocity sensors coupled to the catheter;
a sensor network coupled to the plurality of flow velocity sensors; and
a processor coupled to the sensor network;
wherein each of the plurality of flow velocity sensors is configured to sense a velocity of a blood flow,
wherein an output of the sensor network is configured to be input into a mathematical model stored in the processor, and
wherein the mathematical model is configured to calculate the flow velocity in the blood vessel where the catheter is located.

2. The arrangement according to claim 1, wherein the sensor network further comprises a pressure sensor, wherein the pressure sensor is configured to sense a pressure within the blood vessel.

3. The arrangement according to claim 1, where the mathematical model comprises or is a function, a polynomial function, a regression model, a lumped parameter model, a decision tree, a random forest, a neural network or a numerical model, wherein the mathematical model is configured to output a velocity vector of the flow velocity.

4. The arrangement according to claim 3, wherein the velocity vector is independent of the catheter orientation.

5. The arrangement according to claim 1, where a quality of the sensed parameters is configured to be evaluated by means of at least one of a regression coefficient, a correlation coefficient, or a fitting coefficient.

6. The arrangement according to claim 1, wherein the mathematical model comprises information about a geometry of the catheter and/or an impact of the catheter on the flow velocity, wherein the information is configured to allow for the mathematical model to compensate for the geometry of the catheter and/or the impact of the catheter on the flow velocity.

7. The arrangement according to claim 1, wherein an output of the mathematical model is signalled to a user.

8. The arrangement according to claim 1, wherein the mathematical model is configured to be tailored to be specific for different blood vessel geometries and flow conditions.

9. The arrangement according to claim 1, wherein the mathematical model is tuned to identify laminar and/or transition and/or turbulent flow regimes within the blood vessel.

10. The arrangement of claim 1, wherein the plurality of flow velocity sensors are hot-wire anemometer sensors.

11. The arrangement of claim 10, wherein each of the plurality of flow velocity sensors are configured to thermally influence at least one other flow velocity sensor in the plurality of flow velocity sensors.

12. The arrangement of claim 1, wherein the mathematical model is a numerical model, and wherein the mathematical model comprises a Navier-Stokes equation, wherein an output of the Navier-Stokes equation is compared with the sensed blood flow velocity, and wherein an index of merit is configured to be calculated based on the comparison.

13. The arrangement of claim 1, wherein the mathematical model is a lumped parameter model, wherein the lumped parameter model comprises or consists of discrete entities configured to approximate the behaviour of the output of the plurality of flow velocity sensors, and wherein the lumped parameter model is defined by:
$$\frac{dQ}{dt} = - h \cdot A ( T ( t ) - {T}_{env} ) = - h \cdot A Δ T ( t ) ,$$
wherein Q is thermal energy in Joules, h is a heat transfer coefficient between the catheter and the blood flow, A is a surface area of the heat transfer, T is a temperature of a surface of the catheter, Tₑₙᵥ is a temperature of the environment and ΔT(t) is a time-dependent thermal gradient between the environment and the catheter.

14. The arrangement of claim 1, further comprising an alarm, wherein the alarm indicates to a user if the blood flow velocity falls outside of a predetermined range.

15. A method for measuring a flow velocity in a blood vessel by an arrangement,
wherein the arrangement comprises:
a catheter configured to be inserted into a blood vessel;
a plurality of flow velocity sensors coupled to the catheter;
a sensor network coupled to the plurality of flow velocity sensors; and
a processor coupled to the sensor network, and
wherein the method comprises:
sensing a velocity of a blood flow of the blood vessel by each of the plurality of flow velocity sensors;
transmitting the sensed velocity by each of the flow velocity sensors to the sensor network;
inputting an output of the sensor network into a mathematical model stored in the processor; and
calculating, by the mathematical model, the flow velocity in a blood vessel where the catheter is located.
